(19) United States
(12) Patent Application Publication
(10) Pub. No.: US 2014/0271633 A1
(43) Pub. Date: Sep. 18, 2014

(54) MAMMALIAN CELL CULTURE PERFORMANCE THROUGH SURFACTANT SUPPLEMENTATION OF FEED MEDIA

(71) Applicant: ABBVIE INC., North Chicago, IL (US)

(72) Inventor: Patrick Hossler, Westborough, MA (US)

(73) Assignee: ABBVIE INC., North Chicago, IL (US)

(21) Appl. No.: 14/209,999

(22) Filed: Mar. 13, 2014

Related U.S. Application Data
(60) Provisional application No. 61/784,890, filed on Mar. 14, 2013.

(51) Int. Cl. C07K 16/24; C12N 5/00

(52) U.S. Cl. CPC C07K 16/241; C12N 5/0018
USPC 424/133.1; 435/69.6; 530/387.3; 435/404

(57) ABSTRACT

The present invention provides methods for increasing cell culture performance through the use of chemically defined feed media (CDFM). In particular, the present invention provides methods for the use of surfactants as supplements to CDFM to allow for higher concentrations of media components and thereby result in increased cell culture performance.

### RELATED APPLICATIONS

This application claims priority to U.S. Provisional Patent Application Ser. No. 61/784,890, filed on Mar. 14, 2013, the entire contents of which are incorporated herein by reference.

### BACKGROUND OF THE INVENTION

The use of chemically defined media in mammalian cell culture techniques is advantageous for many reasons, including, but not limited to, better traceability of raw materials, and better lot-to-lot consistency, which facilitate consistency in process performance. In contrast, the use of undefined, complex media components, such as yeast and soy hydrolysates, contribute to process performance variability, including differences in cell growth, product titer, and product quality attributes. Accordingly, the development and refinement of chemically defined media is particularly important for upstream process development, particularly in light of regulatory concerns and the desire for process robustness.

Chemically defined media, e.g., chemically defined basal media (CDBM) and chemically defined feed media (CDFM), can have, even when completely defined, one hundred or more individual chemical species. Often the relative contributions towards process performance of each of these species are not completely understood. Therefore, it is difficult to predict what effect will be observed for any given addition or removal of a species. The use of concentrated media is a typical approach towards the improvement of cell culture performance. One drawback to this strategy is that the preparation of concentrated feed media is often limited by the solubility limit of the respective media components. Researchers have typically avoided this by adjusting the pH and temperature of the media so as to keep the respective media components in solution. However, eventually even these approaches lose their effectiveness in keeping compounds in solution long enough for practical use of the media in GMP production environments. Thus, there remains a need in the art for methods and compositions that will facilitate enhanced solubility of concentrated media components and which can thereby improve cell culture performance.

### SUMMARY OF THE INVENTION

The present invention provides methods for increasing cell culture performance across distinct chemically defined feed media (CDFM) and/or cell lines. In certain embodiments, the present invention relates to supplementing CDFM with surfactants so that media components, particularly concentrated media components, remain in solution for a longer duration, effectively allowing the use of concentrated feed media, which could not be used otherwise.

In one aspect, the present invention provides methods of increasing cell culture performance. The methods include: (a) culturing a cell line that expresses a protein of interest in a culture media; and (b) supplementing the culture media with a chemically defined feed media (CDFM) comprising a surfactant, wherein the surfactant is present in an amount sufficient to achieve increased cell culture performance, thereby increasing cell culture performance.

In some embodiments, the cell line is selected from the group consisting of Chinese Hamster Ovary (CHO) cells, CHO DUX-B11, CHO-K1, NS0 myeloma cells, CV-1 in Origin carrying SV40 (COS) cells, SP2 cells, human embryonic kidney (HEK) cells, baby hamster kidney (BHK) cells, African green monkey kidney VERO-76 cells, HELA cells, human lung cells (W138), and human hepatoma line (Hep G2). In certain embodiments, the cell line is CHO cells, CHO DUX-B11 cells, or CHO-K1 cells.

In other embodiments, the culture media is selected from the group consisting of Iscove's Modified Dulbecco's Medium (IMDM); IMDM with HEPES and L-Glutamine; IMDM with HEPES and without L-Glutamine; RPMI 1640; RPMI 1640 with L-Glutamine; RPMI 1640 with HEPES, L-Glutamine and/or Penicillin-Streptomycin; Minimal Essential Medium-alpha (MEM-alpha); Dulbecco's Modification of Eagle's Medium (DMEM); DMEM high Glucose with L-Glutamine; DMEM high glucose without L-Glutamine; DMEM low Glucose without L-Glutamine; DMEM:F12 1:1 with L-Glutamine; DME/F12; Basal Medium Eagle with Earle's BSS; GMEM (Glasgow's MEM); GMEM with L-glutamine; Grace's Complete Insect Medium; Grace's Insect Medium without FBS; F-10; F-12; Ham's F-10 with L-Glutamine; Ham's F-12 with L-Glutamine; IPL-41 Insect Medium; L-15 (Leibovitz)(2×) without L-Glutamine or Phenol Red; L-15 (Leibovitz) without L-Glutamine; McCoy's 5A Modified Medium; Medium 199; MEM Eagle without L-Glutamine or Phenol Red (2×); MEM Eagle-Earle's BSS with L-glutamine; MEM Eagle-Earle's BSS without L-Glutamine; MEM Eagle-Hanks BSS without L-Glutamine; NCTC-109 with L-Glutamine; Richter's CM Medium with L-Glutamine; Schneider's Insect Medium; and hydrolysate-containing media.

In certain embodiments, the protein is a therapeutic protein, or therapeutically active fragment thereof. In an exemplary embodiment, the therapeutic protein or therapeutically active fragment thereof is an antibody or antigen-binding fragment thereof. In one embodiment, the antibody is HUMIRA®, or an antigen-binding fragment thereof.

In some embodiments, the surfactant is selected from the group consisting of fatty alcohols; polyoxyethylene glycol octylphenol ethers; and polyoxyethylene glycol sorbitan alkyl esters. In other embodiments, the surfactant is a non-ionic surfactant. In exemplary embodiments, the surfactant is selected from the group consisting of polysorbate 80 (PS80), polysorbate 20 (PS20), and poloxamer 188 (P188). In certain embodiments, the concentration of the surfactant in said CDFM is about 0.0025% to about 0.25% (v/v) of PS80; about 0.0025% to about 0.25% (v/v) of PS20; or about 0.1% to about 5.0% (w/v) of P188. In another embodiment, one or more non-ionic surfactant may be combined in an amount disclosed herein.

In one embodiment, the concentration of the surfactant in the CDFM is about 0.0025% to about 0.25% (v/v) of PS80. For example, the concentration of PS80 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%.

In another embodiment, the concentration of the surfactant in the CDFM is about 0.0025% to about 0.25% (v/v) of PS20. For example, the concentration of PS20 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%.

In a further embodiment, the concentration of the surfactant in the CDFM is about 0.1% to about 5.0% (w/v) of P188. For example, the concentration of P188 (v/v) in CDFM is about 0.1%, 0.3%, 0.5%, 0.7%, 1.0%, 1.2%, 1.5%, 1.7%, 2.0%, 2.2%, 2.5%, 2.7%, 3.0%, 3.2%, 3.5%, 3.7%, 4.0%, 4.2%, 4.5%, 4.7%, 4.9%, or 5.0%.

In certain embodiments, the CDFM is employed at an enriched concentration. In other embodiments, the CDFM is employed at a 2×, 2.5×, 3×, 3.5×, 4×, 4.5×, 5×, 5.5×, 6×, 6.5×, 7×, 7.5×, 8×, 8.5×, 9×, 9.5×, 10×, 12×, 15×, or 20× concentration.

In other embodiments, increased cell performance comprises one or more performance characteristics selected from the group consisting of increased protein yield; increased cell specific productivity; increased protein titer; a decrease in the production of high molecular weight (HMW) species; and an increase in the production of monomeric species. In certain embodiments, said protein yield is increased by about 5%, 10%, 15%, 20%, 25%, 30%, 35%, 40%, 45%, 50%, 55%, 60%, 65%, 70%, 75%, or 80%. In another embodiment, the production of high molecular weight species is decreased by about 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90%.

In some embodiments, the CDFM and/or the culture media is not supplemented with a lipid. In one embodiment, the surfactant inhibits aggregation of an amino acid in the CDFM. In another embodiment, the surfactant does not inhibit aggregation of a lipid in the CDFM.

In another aspect, the present invention provides a protein composition produced by any of the methods described herein. In certain embodiments, the protein is a therapeutic protein or a therapeutically active fragment thereof. In an exemplary embodiment, the therapeutic protein, or therapeutically active fragment thereof, is an antibody, or antigen-binding fragment thereof, for example, is HUMIRA®.

In yet another aspect, the present invention provides a method of treating a subject in need thereof, comprising administering to the subject the composition produced according to the method described herein, thereby treating the subject in need thereof.

In a further aspect, the present invention provides a method of treating a subject having a disorder in which TNF-alpha is detrimental, by administering to the subject the composition produced according to the method described herein, thereby treating the subject having a disorder in which TNF-alpha is detrimental. In certain embodiments, the disorder in which TNFα is detrimental is selected from the group consisting of rheumatoid arthritis (RA), juvenile idiopathic arthritic, psoriatic arthritis, ankylosing spondylitis, Crohn's Disease, ulcerative colitis, plaque psoriasis, active axial spondyloarthritis (active axSpA), and non-radiographic axial spondyloarthritis (nr-axSpA).

In any of the foregoing aspects and embodiments, complex media may be used in place of CDFM. For example, the present invention provides a method of increasing cell culture performance by (a) culturing a cell line that expresses a protein of interest in a culture media; and (b) supplementing the culture media with a complex media comprising a surfactant, wherein the surfactant is present in an amount sufficient to achieve increased cell culture performance, thereby increasing cell culture performance.

In another aspect, the present invention provides a chemically defined feed media (CDFM) comprising a surfactant in an amount sufficient to maintain concentrated media components in solution, e.g., an amount sufficient to reduce amino acid aggregation. In some embodiments, the surfactant is selected from the group consisting of fatty alcohols; polyoxyethylene glycol octylphenol ethers; and polyoxyethylene glycol sorbitan alkyl esters. In other embodiments, the surfactant is a non-ionic surfactant. For example, the surfactant is selected from the group consisting of polysorbate 80 (PS80), polysorbate 20 (PS20), and poloxamer 188 (P188). In certain embodiments, the concentration of the surfactant in the CDFM is about 0.0025% to about 0.25% (v/v) of PS80; about 0.0025% to about 0.25% (v/v) of PS20; or about 0.1% to about 5.0% (w/v) of P188. In another embodiment, one or more non-ionic surfactant may be combined in an amount disclosed herein.

In one embodiment, the concentration of the surfactant in the CDFM is about 0.0025% to about 0.25% (v/v) of PS80. For example, the concentration of PS80 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%.

In another embodiment, the concentration of the surfactant in the CDFM is about 0.0025% to about 0.25% (v/v) of PS20. For example, the concentration of PS20 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%.

In a further embodiment, the concentration of the surfactant in the CDFM is about 0.1% to about 5.0% (w/v) of P188. For example, the concentration of P188 (v/v) in CDFM is about 0.1%, 0.3%, 0.5%, 0.7%, 1.0%, 1.2%, 1.5%, 1.7%, 2.0%, 2.2%, 2.5%, 2.7%, 3.0%, 3.2%, 3.5%, 3.7%, 4.0%, 4.2%, 4.5%, 4.7%, 4.9%, or 5.0%.

In another aspect, the present invention provides an antibody, or antigen-binding portion thereof, e.g., HUMIRA®, wherein the antibody, or antigen-binding portion thereof, is produced from cells grown in a culture media supplemented with a chemically defined feed media (CDFM) comprising a surfactant, and wherein the antibody, or antigen-binding portion thereof, comprises a decrease in high molecular weight (HMW) species by about 0.1%, 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90% relative to the antibody, or antigen-binding portion thereof, when produced from cells grown in the culture media not supplemented with CDFM comprising the surfactant.

In some embodiments, the antibody, or antigen-binding portion thereof, further comprises an increase in monomer species by 0.1%, 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90% relative to the antibody, or antigen-binding portion thereof, when produced from cells grown in the culture media not supplemented with CDFM comprising the surfactant. In an exemplary embodiment, the antibody, or antigen-binding portion thereof, is HUMIRA®. In certain embodiments, the HMW and monomer species are assayed using size exclusion chromatography.

In a further embodiment, the surfactant used to produce the antibody, or antigen-binding portion thereof, is selected from the group consisting of fatty alcohols; polyoxyethylene glycol octylphenol ethers; and polyoxyethylene glycol sorbitan alkyl esters. In some embodiments, the surfactant is a non-ionic surfactant. In an exemplary embodiment, the surfactant is selected from the group consisting of polysorbate 80 (PS80), polysorbate 20 (PS20), and poloxamer 188 (P188). In certain embodiments, the concentration of the surfactant in said CDFM is about 0.0025% to about 0.25% (v/v) of PS80; about 0.0025% to about 0.25% (v/v) of PS20; or about 0.1% to about 5.0% (w/v) of P188.

In another aspect, the present invention provides a method of treating a subject in need thereof, comprising administering to the subject the antibody, or antigen-binding fragment thereof, as described herein, thereby treating the subject in need thereof.

In a further aspect, the present invention provides a method of treating a subject having a disorder in which TNF-alpha is detrimental, comprising administering to the subject the antibody, or antigen-binding fragment thereof, as described herein, thereby treating the subject having a disorder in which TNF-alpha is detrimental. In some embodiments, the disorder in which TNFα is detrimental is selected from the group consisting of rheumatoid arthritis (RA), juvenile idiopathic arthritic, psoriatic arthritis, ankylosing spondylitis, Crohn's Disease, ulcerative colitis, plaque psoriasis, active axial spondyloarthritis (active axSpA) and non-radiographic axial spondyloarthritis (nr-axSpA).

### BRIEF DESCRIPTION OF THE DRAWINGS

FIG. 1 depicts a time course profile of concentrated feed media in solution.

FIG. 2 depicts the cell culture performance of Cell Line 1 in CDFM with various concentrations of PS80. (A) Viable cell density (B) Viability (C) Relative harvest titer; the ratio from each experimental condition to the 1×CDFM control (D) Comparative SEC; the subtractive difference in absolute %'s of each SEC species for each experimental condition to the 1×CDFM control (*p<0.05 on marked day or process condition indicating a statistically significant difference compared to 1×CDFM)

FIG. 3 depicts the cell culture performance of Cell Line 1 in CDFM with various concentrations of PS20. (A) Viable cell density (B) Viability (C) Relative harvest titer; the ratio from each experimental condition to the 1×CDFM control (D) Comparative SEC; the subtractive difference in absolute %'s of each SEC species for each experimental condition to the 1×CDFM control (*p<0.05 on marked day or process condition indicating a statistically significant difference compared to 1×CDFM)

FIG. 4 depicts the Cell culture performance of Cell Line 1 in CDFM with various concentrations of P188. (A) Viable cell density (B) Viability (C) Relative harvest titer; the ratio from each experimental condition to the 1×CDFM control (D) Comparative SEC; the subtractive difference in absolute %'s of each SEC species for each experimental condition to the 1×CDFM control (*p<0.05 on marked day or process condition indicating a statistically significant difference compared to 1×CDFM)

FIG. 5 depicts Cell Line 1 performance in bioreactor cultures with 0.01% PS80. (A) Viable cell density (B) Viability (C) Lactate (D) pCO₂ (E) Osmolality (F) Relative harvest titer (G) Relative specific productivity

FIG. 6 depicts Cell Line 2 performance in bioreactor cultures with 0.01% PS80. (A) Viable cell density (B) Viability (C) Lactate (D) pCO₂ (E) Osmolality (F) Relative harvest titer (G) Relative specific productivity

### DETAILED DESCRIPTION OF THE INVENTION

The present methods and compositions are based on the observation that the selective supplementation of surfactants into chemically defined feed media (CDFM) facilitates media components to remain in solution for a longer duration. Accordingly, the present invention relates to supplementing CDFM with surfactants so that media components, particularly concentrated media components, remain in solution for a longer duration, effectively allowing the use of concentrated feed media, which could not be used otherwise.

Surfactants typically cause cell death due to their innate propensity to break apart cell membranes. In certain embodiments, the present method prevents surfactant-mediated cell death by supplementing surfactants at an optimal concentration which does not have an adverse impact on cell growth, while effectively maintaining concentrated media solubility.

In some embodiments, the present method enables the use of concentrated CDFM which significantly improves, for example, protein yield, monoclonal antibody titers, and specific productivity, as well as reduces protein aggregation. Further, as demonstrated herein, the resulting positive impact results directly through the use of the enriched media, and not a result of the surfactants or higher osmolality. The methods of the invention represent a new use of surfactants as feed media supplements to enable the practical use of very concentrated feed media (e.g., 2×CDFM) which would have precipitated out of solution after only a couple of days without the surfactants.

### A. Definitions

As used in the specification and the appended claims, the singular forms “a,” “an” and “the” include plural referents unless the context clearly dictates otherwise. Thus, for example, reference to “a compound” includes mixtures of compounds.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, or within 5-fold, or within 2-fold, of a value.

The term “cells” or “cell line” as used herein refers to a cell population, wild-type or recombinant, which may be cultured (i.e., grown or propagated) according to the methods provided herein. In certain embodiments, the cells or cell lines are capable of producing a recombinant protein of interest. As used herein, the cells or cell lines include those into which a recombinant expression vector has been introduced. Exemplary cells and cell lines are disclosed herein, and are readily recognized by one of ordinary skill in the art.

The term “surfactant” as used herein is known in the art and is generally defined as an agent that reduces the surface tension of liquids and/or solids. For example, a surfactant includes a fatty alcohol (e.g., steryl alcohol), a polyoxyethylene glycol octylphenol ether (e.g., Triton X-100), or a polyoxyethylene glycol sorbitan alkyl ester (e.g., polysorbate 20, 40, 60). In certain embodiments the surfactant is selected from the group consisting of Polysorbate 80 (PS80), polysorbate 20 (PS20), poloxamer 188 (P188). In an exemplary embodiment, the concentration of the surfactant in chemically defined feed media is about 0.0025% to about 0.25% (v/v) of PS80; about 0.0025% to about 0.25% (v/v) of PS20; or about 0.1% to about 5.0% (w/v) of P188.

The term “culture media” (used interchangeably with “culture medium”) as use herein refers to a nutritive composition that aids in sustaining, propagating, and/or differentiating cells. The term “culture media” refers to any medium which is capable of supporting growth, maintenance, propagation, or expansion of cells in an artificial in vitro environment outside of a multicellular organism or tissue. Cell culture medium may be optimized for a specific cell culture use, including, for example, cell culture growth medium which is formulated to promote cellular growth, or cell culture production medium which is formulated to promote recombinant protein production. The culture medium supplies standard inorganic salts, such as zinc, iron, magnesium, calcium and potassium, as well as trace elements, vitamins, an energy source, a buffer system, and essential amino acids. Exemplary culture media include, but are not limited to Iscove's Modified Dulbecco's Medium, RPMI 1640, Minimal Essential Medium-alpha (MEM-alpha), Dulbecco's Modification of Eagle's Medium (DMEM), DME/F12, alpha MEM, Basal Medium Eagle with Earle's BSS, DMEM high Glucose with L-Glutamine, DMEM high glucose without L-Glutamine, DMEM low Glucose without L-Glutamine, DMEM:F12 1:1 with L-Glutamine, GMEM (Glasgow's MEM), GMEM with L-glutamine, Grace's Complete Insect Medium, Grace's Insect Medium without FBS, F-10, F-12, Ham's F-10 with L-Glutamine, Ham's F-12 with L-Glutamine, IMDM with HEPES and L-Glutamine, IMDM with HEPES and without L-Glutamine, IPL-41 Insect Medium, L-15 (Leibovitz) (2×) without L-Glutamine or Phenol Red, L-15 (Leibovitz) without L-Glutamine, McCoy's 5A Modified Medium, Medium 199, MEM Eagle without L-Glutamine or Phenol Red (2×), MEM Eagle-Earle's BSS with L-glutamine, MEM Eagle-Earle's BSS without L-Glutamine, MEM Eagle-Hanks BSS without L-Glutamine, NCTC-109 with L-Glutamine, Richter's CM Medium with L-Glutamine, RPMI 1640 with HEPES, L-Glutamine and/or Penicillin-Streptomycin, RPMI 1640 with L-Glutamine, RPMI 1640 without L-Glutamine, Schneider's Insect Medium, or any other media known to one skilled in the art. Additionally, culture media as described herein include, but are not limited to, chemically defined media, hydrolysate-containing media, and simple media.

The term “chemically defined feed media” (or CDFM), as used herein, refers to media which contain one or more nutrients whose chemical composition and relative concentrations are known, and which is added to the culture media beginning at some time after inoculation. CDFM is sometimes used interchangeably with “concentrated feed media,” “enriched media,” “highly concentrated feed media” or “super concentrated feed media.” CDFM is supplied to the culturing vessel continuously or in discrete increments, to the culture media during culturing, with or without periodic cell and/or product harvest before termination of culture. CDFM may be individually formulated to tailor the needs of a given experimental design and/or desired growth conditions using, for example, a unique blend of amino acids, vitamins, trace minerals, and organic compounds, at enriched amounts to serve as a feed media to cell culture media. Alternatively, commercially available CDFM may be used. Some examples of commercially available CDFM include, but are not limited to, IS CHO Feed-CD (Irvine Scientific), BalanCD™ CHO Feed Medium (1-3) (Irvine Scientific), IS-CHO-V™ (Irvine Scientific), IS-CHO-CD XP™ with Hydrolysate Blend (Irvine Scientific), CHO Feed Bioreactor Supplement (Sigma-Aldrich), CHO CD Efficient Feed™ B nutrient supplement (Life Technologies).

The designation of CDFM as, e.g., 2×, 2.5×, 3×, 3.5×, 4×, 4.5×, or 5× indicates that the particular CDFM concentration employed is a certain-fold more concentrated than a reference, non-concentrated CDFM (i.e., 1×CDFM). Considering the commercially available IS-CHO-V™ CDFM as an example, a 2× or 3× concentration of IS-CHO-V™ may be used, relative to the manufacturer's recommended use at 1× concentration. As a further example, if a unique tailored CDFM formulated at 50 g/L is used as a reference CDFM (i.e., 1×CDFM), then a CDFM formulated at 100 g/L would be designated as 2×CDFM. On the other hand, if a CDFM formulated at 25 g/L is used as a reference CDFM (i.e., 1×CDFM), then a CDFM formulated at 100 g/L would be designated as 4×CDFM. Thus, the 2× or 4× designation is relative to a reference non-concentrated CDFM (i.e., 1×CDFM).

The term “complex media” refers to media containing a hydrolysate or a combination of hydrolysates, i.e., hydrolysates extracted from different sources, as a main ingredient that is added to the cell culture media Like CDFM, the complex media may, for example, be added to the cell culture media according to the methods of the present invention. By way of example, an enriched complex media comprising a surfactant may be added to a cell culture media to increase cell culture performance.

The term “increased cell culture performance” as used herein refers to any desirable increase in the performance of the cell culture as a result of the present method. By way of example, increased cell culture performance includes, but is not limited to, any one or more of the following: increased protein yield; increased antibody titer; increased cell specific productivity; increased maximum cell densities; decrease in high molecular weight species; increase in monomeric species; enhanced cell viability; decreased precipitation in culture media and/or CDFM; enhanced overall product quality as determined by, for example, N-glycan oligosaccharide and size exclusion chromatography; and enhanced overall lot-to-lot consistency.

“Cell specific productivity” or simply “specific productivity” as used herein is measured in units of pg/cell*day, which represents a calculated value based on the experimentally measured expressed protein amount normalized per unit time on a per cell basis.

When using the cell culture techniques of the instant invention, the protein of interest can be produced intracellularly, in the periplasmic space, or directly secreted into the medium. In embodiments where the protein of interest is produced intracellularly, the particulate debris, either host cells or lysed cells (e.g., resulting from homogenization) can be removed by a variety of means, including but not limited to, centrifugation or ultrafiltration. Where the protein of interest is secreted into the medium, supernatants from such expression systems can be first concentrated using a commercially available protein concentration filter, e.g., an Amicon™ or Millipore Pellicon™ ultrafiltration unit.

As used herein, the term “a protein of interest” refers to a protein produced using the methods of the present invention. In certain embodiments the protein is an antibody, e.g., a chimeric antibody, a humanized antibody, a fully human antibody, DVD-Ig, a TVD-IG, or a half-body. In certain embodiments the protein is an antibody of an isotype selected from group consisting of: IgG (e.g., IgG1, IgG2, IgG3, IgG4), IgM, IgA1, IgA2, IgD, or IgE. In certain embodiments the antibody molecule is a full-length antibody (e.g., an IgG1 or IgG4 immunoglobulin) or alternatively the antibody can be a fragment (e.g., an Fc fragment or a Fab fragment). In some embodiments, “protein” also includes, for example, peptides, antigens, toxins, hormones, growth factors, cytokines, clotting factors, enzymes, and fragments thereof.

As used herein, the term “reduce aggregation” or “inhibit aggregation” refer to minimizing or preventing aggregation of, for example, media components or of proteins produced by the methods described herein.

### B. Cells and Cell Culture Techniques

The invention provides methods of cell culture that increase cell culture performance, to enhance, for example, expression of recombinant proteins, e.g., antibodies. The various cell culture media described herein may be used separately or collectively for improved cell culturing, including increased protein production, extended cell longevity, and general increased cell culture performance, as defined herein. The cell culture media used in the present methods may include, in whole or in part, a standard cell culture media, or a modified cell culture media. Modified cell culture media may be derived from standard culture media (also known as basal media) known in the art. Suitable culture media include, but are not limited to Dulbecco's Modified Eagle's Medium (DMEM), DME/F12, Minimal Essential Medium (MEM), Basal Medium Eagle (BME), RPMI 1640, F-10, F-12, alpha-Minimal Essential Medium (alpha-MEM), Glasgow's Minimal Essential Medium (G-MEM), PF CHO, and Iscove's Modified Dulbecco's Medium. Other examples of suitable standard or modified cell culture media are provided herein.

As described herein, in one aspect, chemically defined feed media (CDFM) comprising a surfactant is used together with any one of a variety of cell culture media suitable for the growth of cells. Suitable CDFM to be used in the present methods are commonly known in the art and commercially available. Alternatively, a CDFM may be individually designed and formulated according to the needs of the cell growth conditions, as described herein.

In certain aspects, the cell culture techniques are carried out using CDFM and one or more surfactant supplements or supplement combinations in a culture vessel. In certain embodiments, the cells, CDFM, and surfactant supplement or supplement combinations can be added in any order. For example, the CDFM and surfactant supplement or surfactant supplement combinations may be added to a culture vessel and the cells can then be inoculated into the culture vessel. In another example, cells may be inoculated into the culture vessel containing culture media, and the CDFM and surfactant supplement or surfactant supplement combinations may be added to a culture vessel. The order in which each component is added will depend on the circumstances and will be apparent to those of ordinary skill in the art.

The amount of CDFM to be added to the cell culture media will vary depending on the experimental design, to accommodate different cell lines and different cell culture media. In some embodiments, the feed media volume that is added to the cell culture media is, v/v, 5%, 10%, 12%, 15%, 17%, 20%, 22%, 25%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, or 100% with respect to the initial cell culture media volume.

It will also be appreciated that other types of media such as complex media containing, among others, hydrolysates, may also be used in place of, or in combination with, CDFM according to the present methods. Thus, in some embodiments, the cells, complex media, and surfactant supplement or supplement combinations can be added in any order. For example, the complex media and surfactant supplement or surfactant supplement combinations may be added to a culture vessel and the cells can then be inoculated into the culture vessel. In another example, cells may be inoculated into the culture vessel containing culture media, and the complex media and surfactant supplement or surfactant supplement combinations may be added to a culture vessel. The order in which each component is added will depend on the circumstances and will be apparent to those of ordinary skill in the art.

The selection of cell culture media will depend, in part, on the cell lines used for protein or antibody expression. In certain embodiments, the cells used in the present invention are prokaryote, yeast, or higher eukaryote cells. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In certain embodiments, the cells are eukaryotic microbes such as filamentous fungi or yeast. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

In certain embodiments the cells are derived from multicellular organisms. In particular embodiments, the cells are invertebrate cells from plant and insect cells. Non-limiting examples include cells derived from Spodoptera frugiperda (caterpillar), Aedes aegypti (mosquito), Aedes albopictus (mosquito), Drosophila melanogaster (fruitfly), Bombyx mori, cotton, corn, potato, soybean, petunia, tomato, and tobacco can also be utilized.

In certain embodiments the cells are mammalian cells. For example, the cells are Chinese Hamster Ovary (CHO cells) (including dhfr− CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. Other non-limiting examples of mammalian cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2), the entire teachings of which are incorporated herein by reference.

In particular embodiments, the cells are transformed with expression or cloning vectors for producing products or portions thereof and cultured as appropriate for inducing promoters, selecting transformants, or amplifying the genes encoding the desired sequences. In a particular embodiment, standard molecular biology techniques are used to prepare the recombinant expression vector, transfect the cells, select for transformants, culture the cells and recover the product from the culture medium. In certain embodiments, the cell culture media described herein can be used as culture media for hybridoma cells, monoclonal antibody producing cells, virus-producing cells, transfected cells, cancer cells and/or recombinant peptide producing cells.

The cells of the present invention can be cultured under suitable conditions for suitable periods of time, conditions that depend on the type(s) of cells being cultured and the product being produced. In certain embodiments, the cells are cultured for about two to about fourteen days. In certain embodiments, the cells are cultured from about four to about ten days.

The term “suspension culture” refers to cells in culture in which the majority or all of the cells in culture are present in suspension, and the minority or none of the cells in the culture vessel are attached to the vessel surface or to another surface within the vessel (adherent cells). The “suspension culture” can have greater than about 50%, 60%, 65%, 75%, 85%, or 95% of the cells in suspension, not attached to a surface on or in the culture vessel.

The term “adherent culture” refers to cells in culture in which the majority or all of the cells in culture are present attached to the vessel surface or to another surface within the vessel, and the minority or none of the cells in the culture vessel are in suspension. The “adherent culture” can have greater than 50%, 60%, 65%, 75%, 85%, or 95% of the cells adherent.

The methods of the present invention can include cell culture processes that occur under a variety of environmental conditions. For example, but not by way of limitation, the cells employed in the methods of the instant invention may be cultured while stationary or while shaken/stirred. In certain embodiments, the cells are stirred up to 200 rpm. In certain embodiments, the cells are cultured at a temperature between about 20° C. and about 45° C. In certain embodiments, the cells are cultured at a temperature between about 33° C. and about 37° C. In certain embodiments, the cells are cultured under ambient conditions. In certain embodiments, the cells are cultured in a humidified CO₂ incubator. In certain embodiments, the cells are cultured in a 5% humidified CO₂ incubator. In certain embodiments, the cell culture technique includes providing a barrier between the cells and ambient conditions. In certain embodiments, the barrier is sterile. In certain embodiments, the barrier is a gas permeable, sterile vessel cover. In certain embodiments, the total volume of the combinations of cells, CDFM, and supplements may be from about 0.5 mL to about 2 L. In certain embodiments, the total volume may be from about 1 mL to about 500 mL.

The cell culture techniques of the present methods can be practiced in any suitable culture vessel or devices. For example, in certain embodiments, a culture vessel can refer to a glass, plastic, metal or other container that provides an environment for culturing cells. Non-limiting examples of such culture vessels include incubation vessels, microtiter plates, capillaries, and multi-well plates. In some embodiments, a culture device may refer to, for example, a fermentor type tank culture device, an air lift type culture device, a culture flask type culture device, a spinner flask type culture device, a microcarrier type culture device, a fluidized bed type culture device, a hollow fiber type culture device, a roller bottle type culture device, a packed bed type culture device or any other suitable device known to one skilled in the art.

### C. Supplementation with Surfactants

The instant invention is directed, in part, to methods wherein surfactant supplementation is performed at a concentration and for a duration sufficient to result in increased cell culture performance, e.g., increased cell specific productivity. For example, but not by way of limitation, the addition of about 0.0025% to about 0.25% (v/v) PS80, about 0.0025% to about 0.25% (v/v) PS20; or about 0.1% to about 5.0% (w/v) P188 is sufficient to increase cell culture performance, e.g., cell specific productivity through the direct enabling of the use of concentrated culture media.

By way of example, the concentration of PS80 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%. Similarly, the concentration of PS20 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%. Additionally, the concentration of P188 (v/v) in CDFM is about 0.1%, 0.3%, 0.5%, 0.7%, 1.0%, 1.2%, 1.5%, 1.7%, 2.0%, 2.2%, 2.5%, 2.7%, 3.0%, 3.2%, 3.5%, 3.7%, 4.0%, 4.2%, 4.5%, 4.7%, 4.9%, or 5.0%.

In certain embodiments, the instant invention is directed to methods wherein surfactant supplementation is performed at a concentration and for a duration sufficient to result in increased cell culture performance, e.g., cell specific productivity, while not adversely impacting product quality. For example, but not by way of limitation, surfactant supplementation can be performed at a concentration and for a duration sufficient to enhance cell culture performance (e.g., increased protein yield or cell specific productivity), while not adversely impacting, for example, product glycosylation profiles, cell density, or cell morphology.

Generally, the methods of the invention may use any one or more of an anionic surfactant, a cationic surfactant, a zwitterionic surfactant, or a nonionic surfactant added thereto. Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl glycerol, phosphatidyl inosine, phosphatidylinositol, diphosphatidylglycerol, phosphatidylserine, phosphatidic acid and their salts, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate).

In some embodiments, suitable nonionic surfactants include: glyceryl esters, polyoxyethylene fatty alcohol ethers, polyoxyethylene sorbitan fatty acid esters (polysorbates), polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethyl cellulose, hydroxypropyl cellulose, hydroxypropylmethyl cellulose, noncrystalline cellulose, polysaccharides including starch and starch derivatives such as hydroxyethylstarch (HES), polyvinyl alcohol, and polyvinylpyrrolidone. In certain embodiments, the nonionic surfactant is a polyoxyethylene and polyoxypropylene copolymer and preferably a block copolymer of propylene glycol and ethylene glycol. Such polymers are sold under the tradename POLOXAMER, also sometimes referred to as PLURONIC® F68 or Kolliphor® P188. Among polyoxyethylene fatty acid esters is included those having short alkyl chains. One example of such a surfactant is SOLUTOL® HS 15, polyethylene-660-hydroxystearate.

In some embodiments, suitable cationic surfactants may include, but are not limited to, natural phospholipids, synthetic phospholipids, quaternary ammonium compounds, benzalkonium chloride, cetyltrimethyl ammonium bromide, chitosans, lauryl dimethyl benzyl ammonium chloride, acyl carnitine hydrochlorides, dimethyl dioctadecyl ammomium bromide (DDAB), dioleyoltrimethyl ammonium propane (DOTAP), dimyristoyl trimethyl ammonium propane (DMTAP), dimethyl amino ethane carbamoyl cholesterol (DC-Chol), 1,2-diacylglycero-3-(O-alkyl) phosphocholine, O-alkylphosphatidylcholine, alkyl pyridinium halides, or long-chain alkyl amines such as, for example, n-octylamine and oleylamine.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof. The phospholipid, whether anionic, zwitterionic or a mixture of phospholipids, may be salted or desalted, hydrogenated or partially hydrogenated or natural semi-synthetic or synthetic.

### D. Cell Culture Performance

The methods of the invention increase cell culture performance. As described herein, cell culture performance includes, for example, the production, transcription, translation, post-translational processing, intracellular transport, secretion, and/or turnover of one or more biological and chemical products in cells. Thus, the methods of the invention increase, for example, protein yield, protein (e.g., antibody) titer, cell specific productivity, monomeric species (i.e., reduce overall protein or antibody aggregation), maximum viable cell densities, and cell viability. Similarly, the methods of the invention decrease, for example, high molecular weight species and overall precipitation in culture media and/or CDFM. Moreover, the methods of the invention enhance overall product quality as determined by, for example, N-glycan oligosaccharide and size exclusion chromatography, and enhance overall lot-to-lot consistency.

The term “protein yield” refers to the amount of protein expressed by cultured cells, and can be measured, for example, in terms of grams of protein produced/L medium. If the protein is not secreted by the cells, the protein can be isolated from the interior of the cells by methods known to those of ordinary skill in the art. If the protein is secreted by the cells, the protein can be isolated from the culture medium by methods known to those of ordinary skill in the art. The amount of protein expressed by the cell can readily be determined by those of ordinary skill in the art. In some embodiments, the amount of protein produced can be expressed in terms of cell specific productivity (qₚ). Specific productivity is measured in units of pg/cell*day, a calculated number based on the experimentally measured expressed protein amount normalized per unit time on a per cell basis. In certain embodiments, the protein is a recombinant protein. In one embodiment, the recombinant protein is an antibody or a functional fragment thereof. In an exemplary embodiment, the antibody is HUMIRA®.

In some embodiments, the methods of the invention may be used to increase the yield of biological products, such as proteins and antibodies, produced by the present method. In one embodiment, the methods of the invention can increase the yield of biological products by at least 0.5%, 1.0%, 2.0%, 3.0%, 4.0%, 5.0%, 10%, 15%, 20%, 25%, 30%, 50%, 60%, 70%, 80%, 85%, 90%, 100%, 125%, 150%, 160%, 170%, 200% or 300%. This increase may be the result of the use of, for example, a 2×CDFM supplemented with a surfactant, and is relative to, for example, the yield of the biological products produced in cell culture media without CDFM comprising a surfactant. In other embodiments, this increase is relative to, for example, the yield of the biological products produced in cell culture media with 1×CDFM comprising a surfactant, or a 1×CDFM that does not comprise a surfactant. In one exemplary embodiment, this increase may be the result of the use of feeding a cell culture media with, for example, a 2×CDFM supplemented with a surfactant, and is relative to, for example, the yield of the biological products produced in cell culture media fed with 1×CDFM that is not supplemented with a surfactant. In another embodiment, the biological products produced can be a peptide, such as a therapeutic or diagnostic peptide, polypeptide, protein, monoclonal antibody, immunoglobulin, cytokine (such as interferon), integrin, antigen, growth factor, cell cycle protein, hormone, neurotransmitter, receptor, fusion peptide, blood protein and/or chimeric protein.

In some embodiments, the methods of the invention may be used to increase antibody titer by about 2%, 4%, 6%, 8%, 10%, 12%, 14%, 16%, 18%, 20%, 22%, 24%, 26%, 28%, 30%, 32%, 34%, 36%, 38%, 40%, 43%, 45%, 48%, 50%, 55%, 60%, 65%, 70%, 75%, 80%, 90%, 100%, 125%, 150%, 175%, 200%, 230%, 250%, or 300%. This increase may be the result of the use of, for example, a 2×CDFM supplemented with a surfactant, and is relative to, for example, the titer of the antibody produced in cell culture media without CDFM comprising a surfactant. In other embodiments, this increase is relative to, for example, the titer of the antibody produced in cell culture media with 1×CDFM comprising a surfactant, or a 1×CDFM that does not comprise a surfactant. In one exemplary embodiment, this increase may be the result of feeding a cell culture media with, for example, a 2×CDFM supplemented with a surfactant, and is relative to, for example, the titer of the antibody produced in cell culture media fed with 1×CDFM that is not supplemented with a surfactant.

In some embodiments, the methods of the invention may be used to increase cell specific productivity (qₚ), as described herein, by about 2%, 4%, 6%, 8%, 10%, 12%, 14%, 16%, 18%, 20%, 22%, 24%, 26%, 28%, 30%, 32%, 34%, 36%, 38%, 40%, 43%, 45%, 48%, 50%, 55%, 60%, 65%, 70%, 75%, 80%, 90%, 100%, 125%, 150%, 175%, 200%, 230%, 250%, 300%, 330%, or 350%. This increase may be the result of the use of, for example, a 2×CDFM supplemented with a surfactant, and is relative to, for example, the cell specific productivity produced in cell culture media without CDFM comprising a surfactant. In other embodiments, this increase is relative to, for example, the cell specific productivity produced in cell culture media with 1×CDFM comprising a surfactant, or a 1×CDFM that does not comprise a surfactant. In one exemplary embodiment, this increase may be the result of feeding a cell culture media with, for example, a 2×CDFM supplemented with a surfactant, and is relative to, for example, the cell specific productivity produced in cell culture media fed with 1×CDFM that is not supplemented with a surfactant.

In some embodiments, the present cell culture media and methods may be used to increase maximum viable cell density by about 5%, 10%, 15%, 20%, 30%, 40%, 50%, 60%, 70%, 80%, 90%, 100%, 110%, 120%, 130%, 140%, 150%, 160%, 180%, or 200% as measured over a course of, for example, 14 days. This increase may be the result of the use of, for example, a 2×CDFM supplemented with a surfactant, and is relative to, for example, the maximum viable cell density produced in cell culture media without CDFM comprising a surfactant. In other embodiments, this increase is relative to, for example, the maximum viable cell density produced in cell culture media with 1×CDFM comprising a surfactant, or a 1×CDFM that does not comprise a surfactant. In one exemplary embodiment, this increase may be the result of feeding a cell culture media with, for example, a 2×CDFM supplemented with a surfactant, and is relative to, for example, the maximum viable cell density produced in cell culture media fed with 1×CDFM that is not supplemented with a surfactant.

In some embodiments, the methods of the invention may be used to decrease high molecular weight species by about 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90%. This decrease may be the result of the use of, for example, a 2×CDFM supplemented with a surfactant, and is relative to, for example, the level of high molecular weight species produced in cell culture media without CDFM comprising a surfactant. In other embodiments, this increase is relative to, for example, the level of high molecular weight species produced in cell culture media with 1×CDFM comprising a surfactant, or a 1×CDFM that does not comprise a surfactant. In one exemplary embodiment, this increase may be the result of feeding a cell culture media with, for example, a 2×CDFM supplemented with a surfactant, and is relative to, for example, the level of high molecular weight species produced in cell culture media fed with 1×CDFM that is not supplemented with a surfactant.

The purity of the biological and chemical products may be analyzed using methods well known to those skilled in the art. Non-limiting examples include size-exclusion chromatography, oligosaccharide analysis, Poros™ A HPLC assay, ELISA, western blot analysis, competitive binding assays, direct and indirect sandwich assays, and immunoprecipitation assays. Cell viability values may be measured through trypan blue exclusion, for example.

### E. Composition

Although proteins and, particularly, antibodies have widespread therapeutic applications, a significant limitation of their use is the propensity to self-associate and aggregate. The methods of the invention not only increase cell culture performance to enhance, for example, protein or antibody yield, but also produce protein and antibody products with improved overall product quality as determined by, for example, N-glycan oligosaccharide and size exclusion chromatography. For example, the methods of the invention allow for the production of protein and antibody compositions with reduced high molecular weight species and increased monomeric species.

Thus, the methods of the invention allow for the production of proteins and antibodies with an improved aggregation profile. In one aspect, the present invention provides an antibody, or antigen-binding portion thereof, wherein the antibody, or antigen-binding portion thereof (the “subject protein”), is produced from cells grown in a culture media supplemented with a chemically defined feed media (CDFM) comprising a surfactant, and wherein the antibody, or antigen-binding portion thereof, comprises a decrease in high molecular weight (HMW) species by about 0.1%, 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90% relative to the antibody, or antigen-binding portion thereof (the “control” antibody), when produced from cells grown without, for example, CDFM and/or surfactant.

The term “subject protein” is intended to refer to a protein or antibody product produced according to the methods of the invention. The term “control protein”, as used herein, is intended to refer to a reference protein or antibody composition produced by culturing a cell line in cell culture media which is different from that used to produce the subject protein. For example, a control protein or antibody may be produced using the same host cell line, the same recombinant expression vector, the same cell culture media, same culture vessel, same culture mode, same culture temperature and same pH used to produce the subject protein, but without the same CDFM and/or same surfactant used to produce the subject protein. For example, all other factors being equal, if the subject protein was produced in cell culture media “A” fed with 2×CDFM and 0.01% v/v PS80, the control protein may also be produced from cells grown in culture media “A”, but supplemented with i) 2×CDFM without 0.01% v/v PS80. Apart from the qualitative differences (e.g., difference in level of HMW species), the subject protein and the control protein have the same identity (e.g., HUMIRA® produced in growth conditions of the present invention—“subject antibody”—as compared to HUMIRA® produced without surfactant—“control antibody”).

In certain embodiments, the subject protein or antibody has a decrease in high molecular weight species by about 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90% relative to the control protein or antibody. Additionally, the subject protein or antibody comprises an increase in monomer species by ≦2.6% relative the control protein or antibody. In some embodiments, the subject protein or antibody has an increase in monomer species by about 0.3%, 0.5%, 0.6%, 0.8%, 1.0%, 1.3%, 1.5%, 1.7%, 1.8%, 1.9, 2.0%, 2.1%, 2.2%, 2.3%, 2.4%, 2.5%, 2.6%, 2.7%, 2.8%, 2.9%, 3.0%, 3.2%, 3.5%, 4.0%, 4.5%, 5.0%, 6.0%, 7.0%, 8.0%, 9.0%, 10%, 11%, 13%, 15%, 17%, 20%, 25%, 30%, 35%, 40%, 45%, 50%, 60%, 70%, 80%, or 90% relative to the control protein or antibody. The level of high molecular weight and monomer species can be readily assayed using any of the known methods in the art and include, for example, size exclusion chromatography.

The surfactant used in the present method to produce the protein or antibody may be selected from the group consisting of fatty alcohols; polyoxyethylene glycol octylphenol ethers; and polyoxyethylene glycol sorbitan alkyl esters. In some embodiments, the surfactant is a non-ionic surfactant. In an exemplary embodiment, the surfactant is selected from the group consisting of polysorbate 80 (PS80), polysorbate 20 (PS20), and poloxamer 188 (P188). In certain embodiments, the concentration of the surfactant in said CDFM is about 0.0025% to about 0.25% (v/v) of PS80; about 0.0025% to about 0.25% (v/v) of PS20; or about 0.1% to about 5.0% (w/v) of P188.

By way of example, the concentration of PS80 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%. Similarly, the concentration of PS20 (v/v) in CDFM is about 0.0025%, 0.0035%, 0.0045%, 0.0055%, 0.0065%, 0.0075%, 0.0085%, 0.0095%, 0.01%, 0.015%, 0.02%, 0.025%, 0.03%, 0.04%, 0.05%, 0.06%, 0.07%, 0.08%, 0.09%, 0.10%, 0.12%, 0.14%, 0.16%, 0.18%, 0.20%, 0.21%, 0.22%, 0.23%, 0.24%, or 0.25%. Additionally, the concentration of P188 (v/v) in CDFM is about 0.1%, 0.3%, 0.5%, 0.7%, 1.0%, 1.2%, 1.5%, 1.7%, 2.0%, 2.2%, 2.5%, 2.7%, 3.0%, 3.2%, 3.5%, 3.7%, 4.0%, 4.2%, 4.5%, 4.7%, 4.9%, or 5.0%.

In an exemplary embodiment, the antibody, or antigen-binding portion thereof, produced according to the method of the invention is an anti-TNFalpha antibody. In one embodiment, the anti-TNFalpha antibody is adalimumab, also referred to as HUMIRA®. In certain embodiments, the HUMIRA® produced according to the present invention may has a decreased level of high molecular weight species as compared to the HUMIRA® currently approved and described in the “Highlights of Prescribing Information” for HUMIRA® (adalimumab) Injection (Revised January 2008).

### F. Pharmaceutical Composition

The protein products produced according to the present methods may be prepared and formulated according to the methods known in the art. For example, antibodies produced by the methods of the invention may be formulated with a pharmaceutically acceptable carrier as pharmaceutical (therapeutic) compositions, and may be administered by a variety of methods known in the art. As will be appreciated by the skilled artisan, the route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the proteins or antibodies produced according to the methods herein, and with each other, in a manner such that there is no interaction which would substantially impair the desired pharmaceutical efficacy. In one embodiment, the antibody is an anti-TNFα antibody, or antigen-binding portion thereof.

In one embodiment, a composition produced by the present methods may be formulated with the same or similar excipients and buffers as are present in the commercial adalimumab (HUMIRA®) formulation, as described in the HUMIRA® Prescribing Information, which is expressly incorporated herein by reference. For example, each prefilled syringe of HUMIRA®, which is administered subcutaneously, delivers 0.8 mL (40 mg) of drug product to the subject. Each 0.8 mL of HUMIRA® contains 40 mg adalimumab, 4.93 mg sodium chloride, 0.69 mg monobasic sodium phosphate dehydrate, 1.22 mg dibasic sodium phosphate dehydrate, 0.24 mg sodium citrate, 1.04 mg citric acid monohydrate, 9.6 mg mannitol, 0.8 mg polysorbate 80, and water for Injection, USP. Sodium hydroxide is added as necessary to adjust pH.

The formulations may be present in a form known in the art and acceptable for therapeutic uses. In one embodiment, a formulation of the invention is a liquid formulation. In another embodiment, a formulation of the invention is a lyophilized formulation. In a further embodiment, a formulation of the invention is a reconstituted liquid formulation. In one embodiment, a formulation of the invention is a stable liquid formulation. In one embodiment, a liquid formulation of the invention is an aqueous formulation. In another embodiment, the liquid formulation is non-aqueous. In a specific embodiment, a liquid formulation of the invention is an aqueous formulation wherein the aqueous carrier is distilled water.

In exemplary embodiments, the formulations comprise an antibody in a concentration resulting in a w/v appropriate for a desired dose. The protein or antibody may be present in the formulation at a concentration of about 1 mg/ml to about 500 mg/ml, e.g., at a concentration of at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 200 mg/ml, at least 250 mg/ml, or at least 300 mg/ml.

In a specific embodiment, a formulation of the invention comprises at least about 100 mg/ml, at least about 125 mg/ml, at least 130 mg/ml, or at least about 150 mg/ml of an antibody of the invention.

The formulations described herein may further comprise one or more active compounds as necessary for the particular indication being treated, typically those with complementary activities that do not adversely affect each other. Such additional active compound/s is/are suitably present in combination in amounts that are effective for the purpose intended.

The formulations described herein may include a buffering or pH adjusting agent to provide improved pH control, as well as an excipient (e.g., sugar, salt, surfactant, amino acid, polyol, chelating agent, emulsifier and preservative), an amino acid, pharmaceutically acceptable surfactants, and preservatives, as can be readily appreciated by those of ordinary skill in the art.

The formulation may be a lyophilized formulation. The term “lyophilized” or “freeze-dried” includes a state of a substance that has been subjected to a drying procedure such as lyophilization, where at least 50% of moisture has been removed. Methods of preparing lyophilized compositions as well as methods of reconsitution are well-known in the art.

Therapeutic compositions of the present invention can be formulated for particular routes of administration, such as oral, nasal, pulmonary, topical (including buccal and sublingual), rectal, vaginal and/or parenteral administration. The formulations may conveniently be presented in unit dosage form and may be prepared by any methods known in the art of pharmacy, and include aqueous as well as solid formulations. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. By way of example, in certain embodiments, the antibodies (including antibody fragments) are formulated for intravenous administration. In certain other embodiments, the antibodies (including antibody fragments) are formulated for local delivery to the cardiovascular system, for example, via catheter, stent, wire, intramyocardial delivery, intrapericardial delivery, or intraendocardial delivery.

Formulations of the present invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; US Publication No. 2004-0042972; and 2004-0042971).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The efficient dosages and the dosage regimens for the antibodies of the invention depend on the disease or condition to be treated and can be determined by the persons skilled in the art. One of ordinary skill in the art would be able to determine such amounts based on such factors as the subject's size, the severity of the subject's symptoms, and the particular composition or route of administration selected.

### G. Methods of Treatment

The present compositions and methods may be used to produce protein to be used for any therapeutic purpose in a subject in need thereof.

As used herein, the term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “treatment” or “treat” refers to both therapeutic treatment and prophylactic or preventative measures. Those in need of treatment include those already with the disorder, as well as those in which the disorder is to be prevented.

The term “dosing” or “dose” or “dosage”, as used herein, refers to the administration of a substance (e.g., an antibody of interest, for example, an anti-TNFα antibody, or antigen-binding portion thereof) to achieve a therapeutic objective (e.g., the treatment or amelioration of a symptom of a disease or disorder).

In one embodiment, the therapeutic protein produced according to the present method is an antibody, or antigen-binding portion thereof. In certain exemplary embodiments, the antibody may be an anti-TNFα antibody, or antigen-binding portion thereof. TNFα has been implicated in the pathophysiology of a wide variety of disorders, including sepsis, infections, autoimmune diseases, transplant rejection and graft-versus-host disease (see e.g., Moeller, A., et al. (1990) Cytokine 2:162-169; U.S. Pat. No. 5,231,024 to Moeller et al.; European Patent Publication No. 260 610 B1 by Moeller, A., et al. Vasilli, P. (1992) Annu. Rev. Immunol. 10:411-452; Tracey, K. J. and Cerami, A. (1994) Annu. Rev. Med. 45:491-503). Thus, in one embodiment, the present invention provides methods of producing therapeutic for treating a subject having a disorder in which TNFα activity is detrimental by administering a therapeutically effective amount of an antibody, or antigen-binding portion thereof, thereby treating the TNFα-associated disease or disorder. In one aspect, the TNFα is human TNFα and the subject is a human subject.

As used herein, the term “a disorder in which TNFα activity is detrimental” is intended to include diseases and other disorders in which the presence of TNFα in a subject suffering from the disorder has been shown to be or is suspected of being either responsible for the pathophysiology of the disorder or a factor that contributes to an excerbation of the disorder. Accordingly, a disorder in which TNFα activity is detrimental is a disorder in which inhibition of TNFα activity is expected to alleviate the symptoms and/or progression of the disorder. Such disorders may be evidenced, for example, by an increase in the concentration of TNFα in a biological fluid of a subject suffering from the disorder (e.g., an increase in the concentration of TNFα in serum, plasma, synovial fluid, etc. of the subject), which can be detected, for example, using an anti-TNFα antibody. Disorders in which TNFα activity is detrimental are well known in the art and described in detail in U.S. Pat. No. 8,231,876, the entire contents of which are expressly incorporated herein by reference. Disorders in which TNFα activity is detrimental are also described in “Highlights of Prescribing Information” for HUMIRA® (adalimumab) Injection (Revised January 2008).

In one embodiment, “a disorder in which TNFα activity is detrimental” includes sepsis (including septic shock, endotoxic shock, gram negative sepsis and toxic shock syndrome), autoimmune diseases (including rheumatoid arthritis, rheumatoid spondylitis, osteoarthritis and gouty arthritis, allergy, multiple sclerosis, autoimmune diabetes, autoimmune uveitis, nephrotic syndrome, multisystem autoimmune diseases, lupus (including systemic lupus, lupus nephritis and lupus cerebritis), Crohn's disease and autoimmune hearing loss), infectious diseases (including malaria, meningitis, acquired immune deficiency syndrome (AIDS), influenza and cachexia secondary to infection), allograft rejection and graft versus host disease, malignancy, pulmonary disorders (including adult respiratory distress syndrome (ARDS), shock lung, chronic pulmonary inflammatory disease, pulmonary sarcoidosis, pulmonary fibrosis, silicosis, idiopathic interstitial lung disease and chronic obstructive airway disorders (COPD), such as asthma), intestinal disorders (including inflammatory bowel disorders, idiopathic inflammatory bowel disease, Crohn's disease and Crohn's disease-related disorders (including fistulas in the bladder, vagina, and skin; bowel obstructions; abscesses; nutritional deficiencies; complications from corticosteroid use; inflammation of the joints; erythem nodosum; pyoderma gangrenosum; lesions of the eye, Crohn's related arthralgias, fistulizing Crohn's indeterminant colitis and pouchitis), cardiac disorders (including ischemia of the heart, heart insufficiency, restenosis, congestive heart failure, coronary artery disease, angina pectoris, myocardial infarction, cardiovascular tissue damage caused by cardiac arrest, cardiovascular tissue damage caused by cardiac bypass, cardiogenic shock, and hypertension, atherosclerosis, cardiomyopathy, coronary artery spasm, coronary artery disease, valvular disease, arrhythmias, and cardiomyopathies), spondyloarthropathies (including ankylosing spondylitis, psoriatic arthritis/spondylitis, enteropathic arthritis, reactive arthritis or Reiter's syndrome, and undifferentiated spondyloarthropathies), metabolic disorders (including obesity and diabetes, including type 1 diabetes mellitus, type 2 diabetes mellitus, diabetic neuropathy, peripheral neuropathy, diabetic retinopathy, diabetic ulcerations, retinopathy ulcerations and diabetic macrovasculopathy), anemia, pain (including acute and chronic pains, such as neuropathic pain and post-operative pain, chronic lower back pain, cluster headaches, herpes neuralgia, phantom limb pain, central pain, dental pain, opioid-resistant pain, visceral pain, surgical pain, bone injury pain, pain during labor and delivery, pain resulting from burns, including sunburn, post partum pain, migraine, angina pain, and genitourinary tract-related pain including cystitis), hepatic disorders (including hepatitis, alcoholic hepatitis, viral hepatitis, alcoholic cirrhosis, al antitypsin deficiency, autoimmune cirrhosis, cryptogenic cirrhosis, fulminant hepatitis, hepatitis B and C, and steatohepatitis, cystic fibrosis, primary biliary cirrhosis, sclerosing cholangitis and biliary obstruction), skin and nail disorders (including psoriasis (including chronic plaque psoriasis, guttate psoriasis, inverse psoriasis, pustular psoriasis and other psoriasis disorders), pemphigus vulgaris, scleroderma, atopic dermatitis (eczema), sarcoidosis, erythema nodosum, hidradenitis suppurative, lichen planus, Sweet's syndrome, scleroderma and vitiligo), vasculitides (including Behcet's disease), and other disorders, such as juvenile rheumatoid arthritis (JRA), endometriosis, prostatitis, choroidal neovascularization, sciatica, Sjogren's syndrome, uveitis, wet macular degeneration, osteoporosis, osteoarthritis, active axial spondyloarthritis (active axSpA) and non-radiographic axial spondyloarthritis (nr-axSpA).

In one embodiment, the invention provides a method of administering a compositon comprising an anti-TNFα antibody, or antigen binding portion thereof to a subject such that TNFα activity is inhibited or a disorder in which TNFα activity is detrimental is treated. In one aspect, the TNFα is human TNFα and the subject is a human subject. In one embodiment, the anti-TNFα antibody is adalimumab, also referred to as HUMIRA®.

The proteins produced by the present methods may be administered by a variety of methods known in the art. Exemplary routes/modes of administration include subcutaneous injection, intravenous injection or infusion. In certain aspects, a composition comprising an antibody, or antigen-binding portion thereof, may be orally administered. As will be appreciated by the skilled artisan, the route and/or mode of administration will vary depending upon the desired results.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In certain embodiments, it is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a composition comprising an antibody, or antigen-binding portion thereof, may be 0.01-20 mg/kg, or 1-10 mg/kg, or 0.3-1 mg/kg. In certain specific embodiments, for an anti-TNFα antibody, or antigen-binding portion thereof, such as adalimumab, an exemplary dose is 40 mg every other week. In some embodiments, in particular for treatment of ulcerative colitis or Crohn's disease, an exemplary dose includes an initial dose (Day 1) of 160 mg (e.g., four 40 mg injections in one day or two 40 mg injections per day for two consecutive days), a second dose two weeks later of 80 mg, and a maintenance dose of 40 mg every other week beginning two weeks later. Alternatively, for psoriasis, for example, a dosage can include an 80 mg initial dose followed by 40 mg every other week starting one week after the initial dose.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### EXAMPLES

### A. Materials & Methods

1. Cell Culture

Two recombinant Chinese Hamster Ovary (CHO) cell lines expressing two different humanized monoclonal antibodies were evaluated in two different culture vessels (shaker flasks and laboratory scale bioreactors). Cell Line 1 was of CHO DUX-B11 origin based on a dhfr (dihydrofolate reductase) expression system and Cell Line 2 was of CHO-K1 origin based on the GS (glutamine synthetase) expression system. Both cell lines were cultured in the same chemically defined basal media (CDBM) and feed media (CDFM), with the latter also incorporating surfactants as supplements for evaluation of any potential benefit relative to non-supplemented controls. pH adjustment steps were employed to solubilize the media powder during preparation of the 1× and 2×CDFM, with the surfactants added to the latter to ensure for long-term media component solubility. In preparation of the cultures, the cell lines were serially expanded through separate seed train inoculums to generate enough cells for inoculation. Initial viable cell densities were targeted to the same approximate initial value, and were measured from a representative subset from each shake flask experiment to confirm the target value was approximately achieved. Process conditions utilized during the cultures differed slightly for each cell line as described in Table 1, but were similar when compared between each cell line and their respective non-surfactant supplemented control conditions. The shake flask cultures for Cell Line 1 were harvested on Day 14 post-inoculation or when cell viability dropped below 50%, and the 3 L laboratory-scale bioreactor cultures for Cell Lines 1 and 2 were harvested on Day 15 post-inoculation.

It is to be noted that other types of media, other than 1× or 2×CDFM as exemplified herein, may also be used in the present methods. For example, complex media comprising hydrolysates may also be employed in the methods described herein.

Viable cell density (VCD) and cell viability values were measured through trypan blue exclusion via Cedex automated cell counters (Roche Applied Science, Indianapolis, Ind.), glucose and lactate values were measured with a YSI 2700 (YSI Inc., Yellow Springs, Ohio) and ABL-805 (Radiometer Medical, Denmark) instruments. Offline pH, DO, and pCO₂ measurements were performed on ABL-5 and ABL-805 (Radiometer Medical, Denmark) blood gas analyzers.

TABLE 1
Process Conditions for Studies with CDFM Supplemented with Surfactants

| | | | | Initial | | | | Surfactant | |
|---|---|---|---|---|---|---|---|---|---|
| | | Working | | Culture | DO | Relative | | Concentration in | Feed Schedule |
| Cell | | Volume | pH | Temperature | Setpoint | CDFM | | CDFM | (% v/v): |
| Line | Vessel | (L) | Setpoint | (° C.) | (%) | Conc. | Surfactant | (% v/v, % w/v)^{b} | Process Day |
| 1 | 3 L | 1.5 | 6.9 | 36 | 30 | 1X | N/A | 0 (control) | 3, 5, 7, 3, 3, 3, 3: |
| | Bioreactors | | | | | 2X | PS80 | 0.01 | 3, 5, 7, 9, 10, 11, 12 |
| | Shake | 0.075 | N/A^{a} | 36 | N/A^{a} | 1X | N/A | 0 (control) | 4, 6, 8, 3, 3, 3, 3: |
| | Flasks | | | | | 2X | PS80 PS20 | Various | 2, 4, 6, 8, 9, 10, 12 |
| | | | | | | | P188 | | |
| 2 | 3 L | 1.5 | 7.0 | 36 | 25 | 1X | N/A | 0 (control) | 3, 5, 7, 10, 10: |
| | Bioreactors | | | | | 2X | PS80 | 0.01 | 3, 5, 7, 9, 11 |

^{a}Cultures run in CO₂ incubators at 5% CO₂ in air; pH and DO parameters were not controlled, and thus did not have setpoint values
^{b}PS80 and PS20 expressed in % v/v, P188 expressed in % w/v throughout this paper

2. Protein A Affinity Chromatography

Antibody titers were measured from crude cell culture harvests on a Poros A™ (Life Technologies, Carlsbad, Calif.) affinity column using an Agilent (Santa Clara, Calif.) 1200 Series HPLC operating with a low pH, step elution gradient with detection at 280 nm. Absolute concentrations were assigned with respect to reference standard calibration curves.

Purified antibodies subjected to additional analytical characterization were purified using MabSelect™ Protein A (GE Healthcare, Piscataway, N.J.) using a low pH, step elution gradient, followed by buffer exchange using Corning Lifesciences (Tewksbury, Mass.) Spin Concentrator X UF columns, or Zeba™ desalting spin columns (Thermo Fisher Scientific, Rockford, Ill.) according to the manufacturers' recommended procedures.

3. Charge Heterogeneity Via Imaged Capillary Iso-Electric Focusing (icIEF)

Purified antibody samples from Cell Line 2 were diluted to 1 mg/mL in formulation buffer, followed by dilution in IEF sample buffer. Each sample was vortexed and centrifuged before transfer to a iCE280 Fast IEF Analyzer (ProteinSimple, Santa Clara, Calif.). Sample transfer time was set at 120 seconds. Each sample was pre-focused at 1,500 V for 1 min, then focused at 3,000 V for 8 minutes. Acidic and basic regions were assigned with respect to pI markers, and subsequently quantitated.

4. Size Exclusion Chromatography

Protein A purified antibody samples from Cell Lines 1 and 2 were diluted when necessary to 0.5-5 mg/mL in 1×PBS, and measured on a TSKgel G3000SW_{XL} column (Tosoh Bioscience, South San Francisco, Calif.) using an isocratic gradient on a Shimadzu (Columbia, Md.) SPD-10A VP HPLC, or equivalent, with detection at 280 nm. High molecular weight (HMW), monomer, and low molecular weight (LMW) species were assigned and subsequently quantitated.

5. N-Glycan Oligosaccharide Profiling

Approximately 200 μg of Protein A purified samples from Cell Lines 1 and 2 were treated with N-glycanase at 37° C. overnight to remove the N-glycans from the protein. The protein was precipitated and the supernatant was taken for subsequent chemical derivatization of the reducing end of the released glycans with 2-aminobenzamide (2-AB) dye. Following the derivatization step, the excess labeled was removed using clean up cartridges and the samples were analyzed using normal phase HPLC with fluorescent detection. Mobile phase A was 100% acetonitrile and mobile phase B was 50 mM ammonium formate pH 4.4. The glycan were eluted from a polyamide column (Prozyme, Hayward, Calif.) using a shallow gradient. The labeled glycans were detected using a fluorescence detector with an excitation wavelength of 330 nm and an emission wavelength of 420 nm.

6. Statistics

Experimental results are expressed as mean±SD for those results generated from at least 3 independent cultures. Experimental results are expressed as the mean for those results generated from less than 3 independent cultures. Results were evaluated for statistical significance (when needed) through 2-sided t-tests, with a requirement of p<0.05 relative to the unsupplemented 1×CDFM (non-concentrated feed media) control condition.

### B. Results and Discussion

1. Time Course Profile of Concentrated Media in Solution

CDFM was prepared at a 2× solute concentration, both with and without 0.01% (v/v) PS80. Both media were incubated at room temperature over time and visually inspected for media components precipitating out of solution. The images of these media are shown in FIG. 1. In less than two days, media components started to precipitate out of solution in the 2×CDFM without PS80. The same concentrated media formulated with 0.01% PS80 was able to be kept in solution for almost a week (FIG. 1), and up to a timeframe considerably longer (image not shown). Thus, 0.01% PS80 is sufficient to keep media components in solution at 2× concentration levels long enough for practical use of the media. Analytical characterization was subsequently performed on the identity of the precipitate from the 2×CDFM without PS80. Amino acid analysis facilitated the identification of one key amino acid as enriched in the precipitate. However, this one particular amino acid was likely not the only component that came out of solution in the concentrated feed media. Its decreased concentration in the media was not enough to account for the resulting levels of precipitation observed. It is highly likely that one or more of the media's salts, sugars, vitamins, or trace metals were complexed and contributed to the formation of the precipitated solute.

In the efforts of evaluating the impact of super-concentrated feed media with surfactants on cell culture performance, a series of experiments were performed utilizing two different CHO cell lines expressing two different recombinant humanized antibodies.

2. Cell Culture Performance in Media Supplemented with Various

### Surfactants

Cell Line 1 was evaluated in fed-batch shake flask cultures with different surfactants at varying concentrations (Table 2). The levels at which the surfactants became detrimental to cell culture performance was determined. Additional cultures were also evaluated utilizing excess NaCl supplementation into 1×CDFM to simulate 2×CDFM osmolality levels. Additional cultures were investigated with 1×CDFM supplemented with 0.01% PS80 or 0.01% PS20 to determine if any increases in productivity were caused by the addition of surfactant alone to the feed media.

TABLE 2
Surfactant types and concentrations evaluated in CHO cell
shake flask culture

| | CDFM | | Surfactant |
|---|---|---|---|
| | Relative | Surfactant | Concentration |
| Condition # ^{a} | Concentration | Evaluated | (% v/v, % w/v) |
| 1 | 1X | N/A (control) | N/A (control) |
| 2 | 1X | PS80 | 0.01 |
| 3 | 1X | PS20 | 0.01 |
| 4 | 2X | PS80 | 0.0025 |
| 5 | 2X | PS80 | 0.005 |
| 6 | 2X | PS80 | 0.01 |
| 7 | 2X | PS80 | 0.05 |
| 8 | 2X | PS80 | 0.25 |
| 9 | 2X | PS80 | 1.25 |
| 10 | 2X | PS20 | 0.0025 |
| 11 | 2X | PS20 | 0.005 |
| 12 | 2X | PS20 | 0.01 |
| 13 | 2X | PS20 | 0.05 |
| 14 | 2X | PS20 | 0.25 |
| 15 | 2X | PS20 | 1.25 |
| 16 | 2X | P188 | 0.1^{b} |
| 17 | 2X | P188 | 0.5^{b} |
| 18 | 2X | P188 | 2.5^{b} |
| 19 | 2X | P188 | 5^{b} |
| 20 | 2X | P188 | 10^{b} |

^{a} The condition of 2X CDFM without use of surfactants could not be performed due to low solubility of the media solute at this concentration.
^{b}Concentrations in % w/v

The cell growth, viability, and harvest titer results are shown in FIGS. 2-4. Overall, there was a nominal and statistically significant decrease in cell growth with use of the 2×CDFM, regardless of the surfactant evaluated. This decrease was most likely attributable to the use of the 2×CDFM itself, or the resulting increase in osmolality as a result thereof, rather than through the use of the surfactants directly. This is readily seen in the VCD profiles for the 1×CDFM conditions which were similar regardless of whether a surfactant was present in the feed media or not. See FIGS. 2A, 3A, 4A. In addition, upon excess supplementation of NaCl to 1×CDFM to match the osmolality of 2×CDFM, the VCD profile decreased dramatically in a statistically significant manner. Amongst those cultures which were fed 2×CDFM with PS80 and PS20, the majority of the conditions were indeed able to support comparable cell growth and viability profiles up to a surfactant concentration of 0.25% (v/v). See FIGS. 2A, 2B, 3A, 3B. At a PS80 and PS20 media concentration of 1.25% (v/v) the cultures only lasted for about 2 days before dying, which was very different from the other 2×CDFM cultures, suggesting the surfactants became toxic at this high concentration, and thus the titers were considerably lower as a result.

These lower titer results are in stark contrast to the results demonstrated with the 2×CDFM with PS80 and PS20 concentrations between 0.0025%-0.25% (v/v). Over this range the relative harvest titer increased from 1.0 for the 1×CDFM to a maximum of 1.51 for the 0.05% PS80 supplemented 2×CDFM culture, and a maximum of 1.52 for the 0.01% PS20 supplemented 2×CDFM culture. See FIGS. 2C and 3C. Amongst the PS80 and PS20 supplemented 2×CDFM conditions which facilitated an increased relative harvest titer, there was only at most an 8% difference in their respective higher titers. The enriched feed media was the root cause towards the resulting higher productivity, and not the presence of the surfactant (whose role is to enable the use of the fully dissolved CDFM), or the resulting higher osmolality of the enriched media. This can be clearly seen in the 1×CDFM with surfactant cultures and 1×CDFM with excess NaCl cultures which facilitated a statistically equivalent level of harvest antibody titer relative to the 1×CDFM control cultures. See FIGS. 2C, 3C, 4C.

In the case of P188, the 2×CDFM was not able to be kept in solution for a time duration as long as PS80 and PS20 supplementation, however the experiment was completed before media precipitation occurred. The cell culture performance results closely resembled that of the PS80 and PS20 supplemented 2×CDFM cultures. It was observed that all P188 supplemented cultures had a decreased cell growth and viability profile over time compared to the 1×CDFM cultures, which was statistically significant at the majority of the time points. See FIGS. 4A, 4B. With the exception of the 10% P188 2×CDFM culture, they also outperformed the 1×CDFM culture supplemented with excess NaCl. These results agree with the previous in that they suggest that either the 2×CDFM, or the higher osmolality as a result thereof, are responsible for the decreased cell growth and lower viability trends. Amongst the conditions evaluated, the relative harvest titer increased from 1.0 for the 1×CDFM culture to 1.48 for the 0.5% P188 culture. See FIG. 4C. At P188 concentrations of 5% and 10% there was a slight drop in viability earlier on in the culture and thus the viable cell density profiles trended lower resulting in a harvest titer that was not as high as the other surfactant supplemented 2×CDFM cultures. It is possible that either the P188 concentrations were at a sufficiently high level to cause premature cell death, or the resulting increase in osmolality, (albeit a small increase) shifted the cell growth curve so that it more closely resembles that of the 1×CDFM cultures with excess NaCl.

Interestingly, in all cultures which were fed 2×CDFM, regardless of the surfactant utilized, total HMW levels decreased in a statistically significant manner. See FIGS. 2D, 3D, 4D. This decrease in HMW showed up primarily as additional monomer, indicating an overall improvement in product quality. Upon inspection of the 1×CDFM cultures supplemented with any of the surfactants, it is apparent that there is not much change in HMW levels compared to the 1×CDFM control, suggesting that the surfactants alone are not the reason for the HMW drop in the 2×CDFM cultures. Hence, it is apparent that the use of the enriched feed media itself is responsible for the drop in HMW levels, and the surfactants are not introducing any adverse changes.

These aforementioned results point to the effectiveness of using surfactants as feed media supplements and the direct enabling of highly concentrated feed media for practical use. However, care must be taken to ensure that the proper surfactant is chosen to enable solute solubility for a long enough duration for practical use of the media, and at a working concentration not too high which would decrease overall cell growth.

3. Cell Line 1 Performance in Concentrated CDFM with PS80 at the 3 L Scale

Cell Line 1 was cultured at larger scale in 3 L bioreactors to further ascertain the culture performance in concentrated CDFM with surfactants. The control cultures were fed with media at 1× concentration levels and compared to cultures fed with media at 2× concentration levels supplemented with 0.01% (v/v) PS80. Other than these differences in feed media formulation, all cultures were under identical operating conditions. The viable cell density, viability, lactate, dissolved carbon dioxide (pCO₂), osmolality, titer, and specific productivity (q_{P}) levels are shown in FIG. 5.

Overall, the cell growth profiles were comparable between those cultures fed with 2×CDFM+0.01% PS80 relative to the cultures fed with 1×CDFM. See FIG. 5A. The viability profiles for those cultures fed with 2×CDFM+0.01% PS80 did start trending lower around Day 8 resulting in a final viability at harvest that was 11% lower on average compared to the 1×CDFM conditions. See FIG. 5B. Thus, PS80 does have the capability to decrease the longevity of a particular upstream process, as further evidenced by the aforementioned shake flask results, and care must be taken to balance the increase in culture performance with that of the potential impact on cell growth.

Other metabolic indicators were monitored throughout the duration of the respective cultures. See FIGS. 5C and 5D. pCO₂ and lactate production are direct measures of the respiratory and metabolic activities of cultured cells, respectively. There was no major difference in pCO₂ between the 2×CDFM+0.01% PS80 cultures with that of the 1×CDFM, suggesting no net change in the overall respiratory activity of the cells. There was however a nominal increase in lactate levels in the 2×CDFM+0.01% PS80 conditions, with a peak concentration of 1.6 g/L achieved on Day 4, followed by a duration of net lactate consumption for the remainder of the culture. Final lactate levels at harvest decreased to 0.6 g/L, which was 0.5 g/L higher than the 1×CDFM cultures. The average osmolality of the cultures fed with 2×CDFM+0.01% PS80 was much higher at harvest (401 mOsm/kg) compared to the 1×CDFM cultures (262 mOsm/kg), which is not surprising considering the increased level of media solute added to the reactors through the concentrated feed. See FIG. 5E.

Antibody titers in the 2×CDFM+0.01% PS80 conditions trended higher throughout the duration of the cultures compared to the 1×CDFM cultures. See FIG. 5F. After 15 days in culture, the 2×CDFM conditions had an average relative titer of 1.34 compared to 1.0 for the 1×CDFM condition, a 34% increase. At Day 17 post-inoculation, the titer for one of the 2×CDFM+0.01% PS80 replicate cultures reached an even higher relative titer of 1.62. The mechanism for the increased productivity in the concentrated feed media conditions was primarily due to the increase in cell specific antibody productivity (q_{P}). In the concentrated feed media relative q_{P} increased to 1.81, an 81% increase compared to the 1×CDFM condition. See FIG. 5G.

The product quality of Antibody 1 was also analyzed from the harvest samples of one of the concentrated and non-concentrated feed media cultures (i.e., 1×CDFM). The N-glycan oligosaccharide and SEC results are shown in Table 3. From the table one can see that there was a 2.6% drop in absolute aggregate levels which mostly showed up as monomer from the cell culture fed with 2×CDFM+0.01% PS80. This decrease in aggregate levels is consistent with that reported from the aforementioned statistically significant shake flask results. There was also at most a 1.9% change in either direction amongst the various N-glycans with the surfactant supplemented 2×CDFM cultures. However, these shifts are not considered to be major changes for this particular antibody. In summary, the results from the laboratory-scale bioreactor cultures suggest that the product quality of Antibody 1 derived from the process with 2× concentrated feed with 0.01% PS80 was not adversely impacted compared to the process with non-concentrated feed.

TABLE 3
Product quality differences of Antibody 1 from cultures of Cell Line 1 fed with 2×
CDFM + 0.01% PS80 and 1× CDFM
SEC

| | Difference between 2× and |
|---|---|
| Species | 1× CDFM cultures (%) |
| HMW | −2.6 |
| Monomer | 2.4 |
| LMW | 0.2 |

N-glycan Oligosaccharides^{a}

| | | Difference between 2× and |
|---|---|---|
| Species^{b} | | 1× CDFM cultures (%) |
| NGA2F- GlcNAc | [chemical structure] | 1.3 |
| NGA2F | [chemical structure] | −1.9 |
| NA1F-GlcNAc | [chemical structure] | −0.1 |
| NA1F | [chemical structure] | −0.5 |
| NA2F | [chemical structure] | 0.1 |
| Man 5 | [chemical structure] | 0.8 |
| Man 6 | [chemical structure] | 0.2 |
| Man 7 | [chemical structure] | −0.1 |
| Man 8 | [chemical structure] | 0.2 |
| Man 9 | [chemical structure] | 0 |

^{a}Measure from Day 17 cell culture samples
^{b} [symbol] Mannose [symbol]
Galactose [symbol]
N-acetylglucosamine [symbol]
Fucose

4. Cell Line 2 Performance in Concentrated CDFM with PS80 at the 3 L Scale

In the efforts of evaluating a different cell line expressing a different antibody for its responsiveness towards the use of concentrated CDFM with surfactants, CHO Cell Line 2 was cultured in 3 L laboratory-scale bioreactors to further ascertain the culture performance in concentrated CDFM with surfactants. The control cultures were fed with media at 1× concentration levels and compared to cultures fed with media at 2× concentration levels supplemented with 0.01% (v/v) PS80. Other than these differences in feed media formulation, all cultures were under identical operating conditions. The viable cell density, viability, lactate, pCO₂, osmolality, titer, and specific productivity (q_{P}) levels are shown in FIG. 6.

A higher maximum VCD (up to 17×10⁶ cells/mL) was observed for Cell Line 2 with similar culture duration compared to the results from Cell Line 1. See FIG. 6A. There was a nominal decrease in VCD in the 2×CDFM+0.01% PS80 cultures starting at Day 8 compared to the 1×CDFM cultures. However, this decrease was very slight, and overall, there was no major impact on cell growth, as was the case for Cell Line 1. However, unlike Cell Line 1 there was no measurable impact on the cell viability profiles with both sets of cultures behaving similarly throughout the entire 15 days of culture. See FIG. 6B. At a concentration of 0.01% PS80 it can be presumed that any potential adverse impact through the addition of this surfactant into the cell culture media is minimized.

Other metabolic indicators were monitored throughout the duration of the respective cultures. See FIGS. 6C and 6D. Although pCO₂ profiles trended nominally higher there was no major difference between the 2×CDFM+0.01% PS80 cultures with that of the 1×CDFM, suggesting no net change in the overall respiratory activity of the cells. Like Cell Line 1, there was also a nominal increase in lactate levels in the 2×CDFM+0.01% PS80 conditions, with a peak concentration of 1.6 g/L achieved on Day 4, followed by a duration of net lactate consumption for the remainder of the culture. Final lactate levels at harvest decreased to 0.6 g/L, which was 0.5 g/L higher than the 1×CDFM cultures. The mechanism of the increased lactate is most likely attributable to changes in metabolism caused by components in the concentrated feed, especially excess glucose levels. The average osmolality of the cultures fed with 2×CDFM+0.01% PS80 was much higher at harvest (446 mOsm/kg) compared to the 1×CDFM cultures (336 mOsm/kg). See FIG. 6E.

Antibody titers in the cultures fed with concentrated media trended higher throughout the duration of the cultures compared to the non-concentrated feed media cultures. See FIG. 6F. After 15 days in culture, the 2×CDFM conditions had an average relative titer of 1.5 compared to 1.0 for the 1×CDFM condition. As was the case for Cell Line 1, the mechanism for the increased productivity in the concentrated feed media conditions was primarily due to the increase in cell specific antibody productivity (q_{P}). In the concentrated feed media relative specific productivity increased 2.3-fold compared to the non-concentrated feed media. See FIG. 6G.

The product quality of Antibody 2 was also analyzed from the harvest samples of all of the concentrated and non-concentrated feed media cultures. The N-glycan oligosaccharide, SEC, and charge heterogeneity results are shown in Table 4. From the table one can see that there was a 2.7% drop in absolute aggregate levels in the culture fed with concentrated media, which mostly showed up as additional monomer. The fact that the aggregates decreased for both Cell Line 1 and Cell Line 2 upon exposure to 2×CDFM with PS80 in reactors is consistent with the aforementioned, and statistically significant shake flask results. Amongst the various N-glycans, there was at most a 2.9% change in either direction, which is a measurable change, but not an adverse change for this particular antibody. In addition, upon inspection of the charge heterogeneity results one can see that there was a 3.4% decrease in acidic species, with the majority of the difference showing up as additional basic species at 2.7%. These results are also not considered major changes for this particular antibody. In conclusion, the aforementioned results suggest that the product quality of Antibody 2 derived from the process with 2× concentrated feed with 0.01% PS80 was not adversely impacted compared to the process with non-concentrated feed.

TABLE 3
Product quality differences of Antibody 2 from cultures of Cell Line 2 fed with 2×
CDFM + 0.01% PS80 and 1× CDFM
SEC

| | Difference between 2× and |
|---|---|
| Species | 1× CDFM cultures (%) |
| HMW | −2.7 |
| Monomer | 2.8 |
| LMW | −0.2 |

N-glycan Oligosaccharides^{a}

| Species^{b} | | |
|---|---|---|
| NGA2F- GlcNAc | [chemical structure] | 0.8 |
| NGA2F | [chemical structure] | 2.2 |
| NA1F- GlcNAc | [chemical structure] | −0.3 |
| NA1F | [chemical structure] | −2.9 |
| NA2F | [chemical structure] | −0.2 |
| Man 5 | [chemical structure] | 0 |
| Man 6 | [chemical structure] | −0.1 |
| Man 7 | [chemical structure] | −0.1 |
| Man 8 | [chemical structure] | 0 |
| Man 9 | [chemical structure] | 0 |

Charge Heterogeneity

| | Difference between 2× and |
|---|---|
| Species | 1× CDFM cultures (%) |
| Acidic | −3.4 |
| Main | 0.7 |
| Basic | 2.7 |

^{a}Measure from Day 17 cell culture samples
^{b} [symbol] Mannose [symbol]
Galactose [symbol]
N-acetylglucosamine [symbol]
Fucose

The cumulative results suggest that 2×CDFM made practical through supplementation of 0.01% PS80, resulted in a nominal impact on the resulting cell growth and cell viability profiles compared to 1×CDFM control cultures. However, there was a dramatic increase in final harvest titers due primarily to the increase in cellular specific productivity. Hence, there appears to be a common response regardless of the expression system, antibody expressed, or culture vessel, in that the use of concentrated media feeds rendered practical through the supplementation of surfactants such as PS80, facilitates an improvement in mammalian cell culture performance.

### C. Conclusion

In the present work, select surfactants were evaluated for their potential role for the enabling of concentrated CDFM, the use of which was shown to significantly improve monoclonal antibody titers. The use of surfactants such as PS20 and PS80 in the biopharmaceutical industry is not without precedent, since they are often utilized in drug substance formulations, where their presence is typically warranted to preclude protein particle formation. The aforementioned results highlight a new use of surfactants as feed media supplements to enable the practical use of very concentrated feed media which would have precipitated out of solution after only a couple days without the surfactants. Surfactants have an obvious role towards cell death due to their innate propensity to break apart cell membranes. The present method strikes a balance towards preventing this by using dilute concentrations which did not have an adverse impact on cell growth, but did have a very effective role at maintaining concentrated media solubility. Surfactant concentration of 0.25% (v/v) in the feed media was found to be the limit before an obvious impact on cell death was facilitated. Utilizing a much lower concentration of 0.01% (v/v) in 2×CDFM in laboratory-scale bioreactor cultures demonstrated only a nominal impact on cell growth and viability profiles. However, the resulting positive impact on, e.g., titers and specific productivity was quite significant. It was further found in this study that this increase in antibody productivity was through the use of the enriched media directly, and not through the associated higher osmolality.

In both cases of Cell Line 1 and Cell Line 2, 2×CDFM with surfactants did not adversely impact product quality. Although the change was slight and close to the historically reported levels, the 2×CDFM with surfactants actually improved and reduced overall aggregation levels. It is apparent from the results however, that it was the enriched media, and not the surfactants themselves, which provided for this benefit.

In summary, the aforementioned top-down media design approach was effective in providing an improved cell culture process in a very rapid fashion with multiple mammalian cell lines and expression systems. The approach is a great fit towards early-stage projects where upstream development timing is typically essential for the overall project timelines, coupled to the fact that very early-stage cell lines are frequently not the best producers. Indeed, the use of surfactants as supplements towards CDFM improvement may find numerous applications in early-stage process development where the speed of providing a sufficient amount of recombinant protein product to the clinic is on the critical path towards project advancement.

All patents, patent applications, publications, product descriptions and protocols, cited in this specification are hereby incorporated by reference in their entirety.

While it will be apparent that the invention herein described is well calculated to achieve the benefits and advantages set forth above, the present invention is not to be limited in scope by the specific embodiments described herein. It will be appreciated that the invention is susceptible to modification, variation and change without departing from the spirit thereof.

What is claimed is:

1. A method of increasing cell culture performance, the method comprising:
(a) culturing a cell line that expresses a protein of interest in a culture media; and
(b) supplementing said culture media with a chemically defined feed media (CDFM) comprising a surfactant,
wherein the surfactant is present in an amount sufficient to achieve increased cell culture performance, thereby increasing cell culture performance.

2. The method of claim 1, wherein the cell line is selected from the group consisting of Chinese Hamster Ovary (CHO) cells, CHO DUX-B11, CHO-K1, NS0 myeloma cells, CV-1 in Origin carrying SV40 (COS) cells, SP2 cells, human embryonic kidney (HEK) cells, baby hamster kidney (BHK) cells, African green monkey kidney VERO-76 cells, HELA cells, human lung cells (W138), and human hepatoma line (Hep G2).

3. The method of claim 2, wherein the cell line is CHO cells, CHO DUX-B11 cells, or CHO-K1 cells.

4. The method of claim 1, wherein the culture media is selected from the group consisting of Iscove's Modified Dulbecco's Medium (IMDM); IMDM with HEPES and L-Glutamine; IMDM with HEPES and without L-Glutamine; RPMI 1640; RPMI 1640 with L-Glutamine; RPMI 1640 with HEPES, L-Glutamine and/or Penicillin-Streptomycin; Minimal Essential Medium-alpha (MEM-alpha); Dulbecco's Modification of Eagle's Medium (DMEM); DMEM high Glucose with L-Glutamine; DMEM high glucose without L-Glutamine; DMEM low Glucose without L-Glutamine; DMEM:F12 1:1 with L-Glutamine; DME/F12; Basal Medium Eagle with Earle's BSS; GMEM (Glasgow's MEM); GMEM with L-glutamine; Grace's Complete Insect Medium; Grace's Insect Medium without FBS; F-10; F-12; Ham's F-10 with L-Glutamine; Ham's F-12 with L-Glutamine; IPL-41 Insect Medium; L-15 (Leibovitz) (2×) without L-Glutamine or Phenol Red; L-15 (Leibovitz) without L-Glutamine; McCoy's 5A Modified Medium; Medium 199; MEM Eagle without L-Glutamine or Phenol Red (2×); MEM Eagle-Earle's BSS with L-glutamine; MEM Eagle-Earle's BSS without L-Glutamine; MEM Eagle-Hanks BSS without L-Glutamine; NCTC-109 with L-Glutamine; Richter's CM Medium with L-Glutamine; Schneider's Insect Medium; and hydrolysate-containing media.

5. The method of claim 1, wherein the protein is a therapeutic protein, or therapeutically active fragment thereof.

6. The method of claim 5, wherein the therapeutic protein, or therapeutically active fragment thereof, is an antibody or antigen-binding fragment thereof.

7. The method of claim 6, wherein the antibody is HUMIRA®.

8. The method of claim 1, wherein the surfactant is selected from the group consisting of fatty alcohols; polyoxyethylene glycol octylphenol ethers; and polyoxyethylene glycol sorbitan alkyl esters.

9. The method of claim 1, wherein the surfactant is a non-ionic surfactant.

10. The method of claim 9, wherein the surfactant is selected from the group consisting of polysorbate 80 (PS80), polysorbate 20 (PS20), and poloxamer 188 (P188).

11. The method of claim 9, wherein the concentration of the surfactant in said CDFM is about 0.0025% to about 0.25% (v/v) of PS80; about 0.0025% to about 0.25% (v/v) of PS20; or about 0.1% to about 5.0% (w/v) of P188.

12. The method of claim 1, wherein increased cell performance comprises one or more performance characteristics selected from the group consisting of increased protein yield; increased cell specific productivity; increased protein titer; a decrease in the production of high molecular weight (HMW) species; and an increase in the production of monomeric species.

13. The method of claim 12, wherein said protein yield is increased by about 80%.

14. The method of claim 12, wherein the production of high molecular weight species is decreased by about 2.6%.

15. The method of claim 1, wherein the CDFM and/or the culture media is not supplemented with a lipid.

16. The method of claim 1, wherein said surfactant inhibits aggregation of an amino acid in said CDFM.

17. The method of claim 1, wherein said surfactant does not inhibit aggregation of a lipid in said CDFM.

18. A protein composition produced by the method of claim 1.

19. The composition of claim 18, wherein the protein is a therapeutic protein or a therapeutically active fragment thereof.

20. The composition of claim 19, wherein the therapeutic protein, or therapeutically active fragment thereof, is an antibody, or antigen-binding fragment thereof.

21. The composition of claim 20, wherein the antibody, or antigen-binding fragment thereof, is HUMIRA®.

22. A method of treating a subject in need thereof, comprising administering to the subject the composition produced according to the method of claim 1, thereby treating the subject in need thereof.

23. A method of treating a subject having a disorder in which TNF-alpha is detrimental, comprising administering to the subject the composition produced according to the method of claim 1, thereby treating the subject having a disorder in which TNF-alpha is detrimental.

24. The method of claim 23, wherein the disorder in which TNFα is detrimental is selected from the group consisting of rheumatoid arthritis (RA), juvenile idiopathic arthritic, psoriatic arthritis, ankylosing spondylitis, Crohn's Disease, ulcerative colitis, plaque psoriasis, active axial spondyloarthritis (active axSpA) and non-radiographic axial spondyloarthritis (nr-axSpA).

25. A chemically defined feed media (CDFM) comprising a surfactant in an amount sufficient to reduce amino acid aggregation.

26. The CDFM of claim 25, wherein the surfactant is selected from the group consisting of fatty alcohols; polyoxyethylene glycol octylphenol ethers; and polyoxyethylene glycol sorbitan alkyl esters.

27. The CDFM of claim 25, wherein the surfactant is a non-ionic surfactant.

28. The CDFM of claim 27, wherein the surfactant is selected from the group consisting of polysorbate 80 (PS80), polysorbate 20 (PS20), and poloxamer 188 (P188).

29. The CDFM of claim 28, wherein the concentration of the surfactant in said CDFM is about 0.0025% to about 0.25% (v/v) of PS80; about 0.0025% to about 0.25% (v/v) of PS20; or about 0.1% to about 5.0% (w/v) of P188.

30. An antibody, or antigen-binding portion thereof, wherein said antibody, or antigen-binding portion thereof, is produced from cells grown in a culture media supplemented with a chemically defined feed media (CDFM) comprising a surfactant, and wherein the antibody, or antigen-binding portion thereof, comprises a decrease in high molecular weight (HMW) species by about 2.6% relative to said antibody, or antigen-binding portion thereof, when produced from cells grown in the culture media not supplemented with CDFM comprising the surfactant.

31. The antibody, or antigen-binding portion thereof, of claim 30, further comprising an increase in monomer species by ≦2.6% relative to said antibody, or antigen-binding portion thereof, when produced from cells grown in the culture media not supplemented with CDFM comprising the surfactant.

32. The antibody, or antigen-binding portion thereof, of claim 30, wherein said antibody is HUMIRA®.

33. The antibody, or antigen-binding portion thereof, of claim 30 or 31, wherein HMW and monomer species are assayed using size exclusion chromatography.

34. The antibody, or antigen-binding portion thereof, of claim 30, wherein the surfactant is selected from the group consisting of fatty alcohols; polyoxyethylene glycol octylphenol ethers; and polyoxyethylene glycol sorbitan alkyl esters.

35. The antibody, or antigen-binding portion thereof, of claim 30, wherein the surfactant is a non-ionic surfactant.

36. The antibody, or antigen-binding portion thereof, of claim 35, wherein the surfactant is selected from the group consisting of polysorbate 80 (PS80), polysorbate 20 (PS20), and poloxamer 188 (P188).

37. The antibody, or antigen-binding portion thereof, of claim 30, wherein the concentration of the surfactant in said CDFM is about 0.0025% to about 0.25% (v/v) of PS80; about 0.0025% to about 0.25% (v/v) of PS20; or about 0.1% to about 5.0% (w/v) of P188.

38. A method of treating a subject in need thereof, comprising administering to the subject the antibody, or antigen-binding fragment thereof, according to claim 30, thereby treating the subject in need thereof.

39. A method of treating a subject having a disorder in which TNF-alpha is detrimental, comprising administering to the subject the antibody, or antigen-binding fragment thereof, according to claim 30, thereby treating the subject having a disorder in which TNF-alpha is detrimental.

40. The method of claim 39, wherein the disorder in which TNFα is detrimental is selected from the group consisting of: rheumatoid arthritis (RA), juvenile idiopathic arthritic, psoriatic arthritis, ankylosing spondylitis, Crohn's Disease, ulcerative colitis, plaque psoriasis, active axial spondyloarthritis (active axSpA) and non-radiographic axial spondyloarthritis (nr-axSpA).
